(19) United States
(12) Patent Application Publication
(10) Pub. No.: US 2024/0115731 A1
(43) Pub. Date: Apr. 11, 2024

(54) METHOD OF DELIVERING MRNA IN VIVO

(71) Applicant: PCI Biotech AS, Oslo (NO)

(72) Inventor: Anders HØGSET, Oslo (NO)

(73) Assignee: PCI Biotech AS, Oslo (NO)

(21) Appl. No.: 18/276,102

(22) PCT Filed: Feb. 8, 2022
(86) PCT No.: PCT/EP2022/053022

§ 371 Date: Aug. 7, 2023

(30) Foreign Application Priority Data
Feb. 8, 2021 (GB) 2101726.4

(51) Int. Cl. A61K 48/00; A61K 41/00

(52) U.S. Cl. CPC A61K 48/0016; A61K 41/0071

(57) ABSTRACT

The present invention concerns an in vivo method for introducing a naked mRNA molecule into the cytosol of a cell(s) in a subject, by the use of photochemical internalization, wherein the photosensitising agent is a sulphonated meso-tetraphenyl chlorin, sulfonated tetraphenylporphine or a di- or tetrasulfonated aluminium phthalocyanine used in an amount of 0.000001-0.001 μg and the cells(s) are contacted with the mRNA molecule and photosensitising agent for 30 seconds to 10 minutes before irradiation of the cell(s). The method may be used to express a polypeptide in the subject. The method is also directed to pharmaceutical compositions containing the photosensitising agents and the mRNA and uses of the molecules in therapy, e.g. to treat or prevent cancer or an infection.

The present invention relates to a method for introducing mRNA into the cytosol of cells in vivo, using a photosensitising agent and irradiation of the cells with light of a wavelength effective to activate the photosensitising agent, and to the use of this method for expressing polypeptides in the cell, e.g. in therapeutic methods. The method is achieved using very low concentrations of the photosensitising agent and short contact times of the active ingredients with the cells before irradiation is performed.

mRNA offers the potential as a drug class for various therapeutic purposes including prophylactic and therapeutic vaccination. mRNA-based therapeutics can, in principle, also be used in the treatment of a very large variety of diseases, e.g. in immunotherapy of cancer and other diseases, in regenerative medicine (e.g. CVD, neurodegenerative diseases), cancer (other than immunotherapy), acute infectious diseases and many others. However, the widespread use of mRNA for these purposes has so far been severely hindered by problems with delivering the functional mRNA into cells.

Carriers have been used to attempt to overcome this issue. Among synthetic carriers, the most common way to deliver mRNA molecules has been by the use of cationic lipids (Feigner and Ringold, 1989, Nature 337, 387-388; Malone et al., 1989, Proc Natl Acad Sci USA 86, 6077-6081; Lu et al., 1994, Cancer Gene Ther 1, 245-252; Kariko et al., 1999, Gene Ther 6, 1092-1100; Hecker et al., 2001, Mol Ther 3, 375-384). In contrast, there are only a few examples of the use of polycations, such as DEAE-dextran (Malone et al. 1989, supra), poly(-lysine) (Fisher and Wilson 1997, Biochem J 321 (Pt 1), 49-58), dendrimers (Nair et al. 1998, Nat Biotechnol 16, 364-369) and polyethylenimine (Bettinger et al. 2001, Nucleic Acids Res 29, 3882-389). The most successfully employed lipid vehicle for mRNA delivery is lipofectamine. However, toxic reactions have been observed using lipofectamine in vivo limiting the potential of this agent for clinical applications.

However, there remains a need for methods of delivering mRNA in vivo which are safe, controlled and direct the mRNA to the required location. WO2019/145419, which is incorporated herein by reference, teaches that naked mRNA may be delivered in vivo in a method which does not rely on carriers but instead uses a photochemical internalisation method to allow controlled and timed release of the mRNA to the site of interest. This method provided superior in vivo delivery of mRNA to target cells relative to the use of lipofectamine as the gold standard for mRNA delivery and is suitable for various therapeutic applications. Comparable efficacy was shown using intratumoural and intradermal administration.

Photochemical internalization (PCI) is a strategy based on light-induced rupture of endocytic membranes, triggered by the use of a photosensitising compound that localizes in these membranes (Berg et al., 1999, Cancer Res 59, 1180-1183). Upon illumination, the photosensitising compound initiates an oxidative process by creating reactive oxygen species (ROS) that destroys the membrane.

PCI methods provide a mechanism for introducing molecules into the cytosol of a cell in a manner which does not result in widespread cell destruction or cell death if the methodology is suitably adjusted to avoid excessive toxic species production, e.g. by lowering illumination times or photosensitiser dose. The basic method of photochemical internalisation (PCI), is described in WO 96/07432 and WO 00/54802, which are incorporated herein by reference. In such methods, the molecule to be internalised (which in the present invention would be the mRNA molecule), and a photosensitising agent are brought into contact with a cell. The photosensitising agent and the molecule to be internalised are taken up into a cellular membrane-contained subcompartment within the cell, i.e. they are endocytosed into an intracellular vesicle (e.g. a lysosome or endosome). On exposure of the cell to light of the appropriate wavelength, the photosensitising agent is activated which directly or indirectly generates reactive oxygen species which disrupt the intracellular vesicle's membranes. This allows the internalized molecule to be released into the cytosol. It was found that in such a method the functionality or the viability of the majority of the cells was not deleteriously affected.

The PCI strategy has been used to deliver a variety of molecules into cytosol in vitro, e.g. siRNA molecules (Boe et al., 2007, Oligonucleotides 17, 166-173; Oliveira et al., 2007, Biochim Biophys Acta 1768, 1211-1217). In vivo, the effect of PCI-mediated therapy on tumour treatment has been documented with bleomycin (Berg et al., 2005, Clin Cancer Res 11, 8476-8485), the protein toxin gelonin (Selbo et al., 2001, Int J Cancer 92, 761-766) and with a plasmid encoding a therapeutic gene (Ndoye et al., 2006 Mol Ther 13, 1156-1162). PCI has been developed for use to treat several types of tumours (Selbo et al., 2010, J. Control. Release, 148(1), 2-12; and Høgset A, et al., 2004, Adv. Drug Deliver. Rev., 56(1), 95-115), and was investigated in a phase I clinical study using bleomycin as the active agent (Sultan et al., 2016, Lancet Oncol. 17(9):1217-1229). PCI is currently in clinical development for the treatment of bile duct cancer using gemcitabine as the active agent (ClinicalTrials.gov ID: NCT01900158).

PCI has been used for delivery of oligonucleotides (Høgset et al., 2004, Adv Drug Deliv Rev, 56, 95-115). Prior to WO2019/145419, whilst mRNA and related molecules such as siRNA had been internalized with PCI, carriers had been used (WO2008/007073 and Bøe and Hovig, 2013, Methods Mol. Biol., 969: 89-100).

WO2019/145419 provides a specific protocol for light-induced mRNA delivery resulting in site-specific protein production. It was shown that potent light-induced protein production was achievable by combining mRNA transfection and PCI. The method has the advantage that it is controllable in a time- and site-specific manner. Furthermore, the method avoids the use of transfection agents and the side effects caused by those agents.

WO2019/145419 is concerned with the use of a photosensitising agent (selected from a sulphonated meso-tetraphenyl chlorin, sulfonated tetraphenylporphine or a di- or tetrasulfonated aluminium phthalocyanine, also referred to the photosensitiser herein) in an amount of 0.0001 to 1 μg. In these methods, the photosensitiser and the mRNA are brought into contact with the subject's cells before the subject's cells are irradiated to activate the photosensitiser. The period of contact, i.e. from contact of the agents with the cells (after administration) to irradiation is preferably from 45 to 90 minutes, to allow uptake of the agents, though longer administration times may be used.

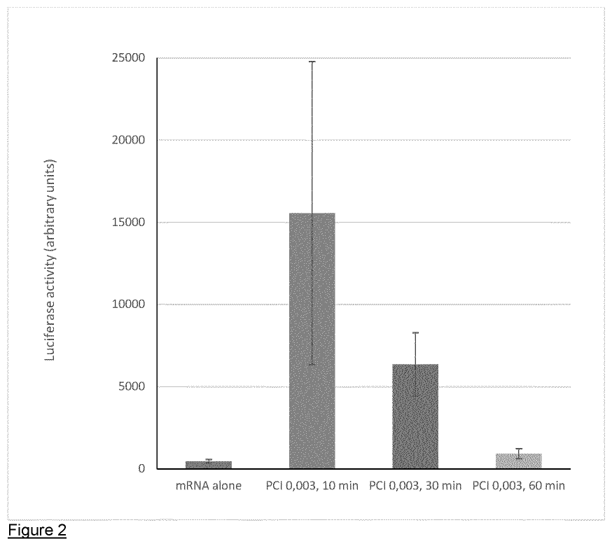

Surprisingly it has now been found that the duration of the contacting step in which both the photosensitiser and mRNA are in contact with the cells, may be drastically reduced to as little as 30 seconds and the photosensitiser concentration may also be drastically reduced without affecting the efficacy of the method. Indeed, superior results have been observed as shown in the Examples (see e.g. FIG. 2). The light dose does not need to be increased to offset the reduced doses or contact time. This is particularly surprising given that it would have been expected that either one of these changes would have led to reduced efficacy.

Prior to the present invention it was believed that sufficient time had to be allowed after administration of the photosensitiser for it to be taken up into intracellular compartments such that when activated by irradiation it would destroy those compartments allowing release of molecules in those compartments (or entering those compartments) into the cytosol. However, a much shorter contacting step (i.e. the time between contact of the cells with the photosensitiser and the mRNA and irradiation) of as little as 30 seconds has been found to be effective. Whilst not wishing to be bound by theory it is believed that the activation of the photosensitiser may be effective not just on the intracellular compartments but also on the plasma membrane making the plasma membrane more permeable to mRNA molecules, whilst retaining the viability of the cells. It is surprising not only that this shorter contacting step is effective, but that it actually improves uptake of mRNA molecules.

The method of the invention has a number of advantages. It is not a complex method and may be used with a variety of mRNA molecules and target cells/locations. Furthermore, the timing and location of irradiation to release the molecules may be controlled such that they are released only at the time and location that is desired to achieve the required effects. As such, exposure of cells to the various components is minimised, and undesirable side effects are minimised. This is in contrast to the standard techniques for mRNA delivery, where it is not possible to control the timing and location of the release of the various components without the use of targeting agents (which add a further level of complexity). In addition, very low doses of the photosensitiser and light doses may be used thereby avoiding side-effects in the treated subject.

As described in the Examples herein, very low levels of photosensitiser (as much as 7×10⁶ times lower than in standard protocols and up to 300 times lower than the amounts used in WO2019/145419) are used to achieve mRNA delivery.

Thus, in a first aspect, the invention provides an in vivo method for introducing an mRNA molecule into the cytosol of a cell(s) in a subject, said method comprising
i) contacting said cell(s) with an mRNA molecule and a photosensitising agent, wherein said mRNA is naked, and
ii) irradiating the cell(s) with light of a wavelength effective to activate the photosensitising agent,
   wherein said photosensitising agent is a sulphonated meso-tetraphenyl chlorin, sulfonated tetraphenylporphine or a di- or tetrasulfonated aluminium phthalocyanine used in an amount of 0.000001 to 0.001 μg and said contacting step is performed for 30 seconds to 10 minutes. Once activated, intracellular compartments within said cell containing said photosensitising agent release mRNA contained in (or which will enter) these compartments into the cytosol where the mRNA may be expressed. In addition, mRNA may be allowed to enter the cytosol directly through the plasma membrane by the action of the photosensitising agent on that membrane when activated.

In a preferred aspect, the photosensitizing agent (particularly TPCS₂ₐ) is used in an amount of 0.000003 to ≤0.0005 μg (or 0.000003 to ≤1.0001, 0.000001 to ≤1.0003 μg, 0.000001 to ≤0.0001 μg, or 0.0001 to ≤1.0003 μg) and the contacting step is performed for 30 seconds to 1, 2, 3, 4 or 5 minutes (e.g. 30-60 seconds) and preferably the cell(s) is irradiated with a light dose of 0.3 to 3 J/cm². Preferably administration is intradermal or intramuscular.

The definitions and preferred aspects defining the methods of the invention similarly apply to uses described herein.

As referred to herein said “mRNA” molecule is a polymer of ribonucleotides, each containing the sugar ribose in association with a phosphate group and a nitrogenous base (typically, adenine, guanine, cytosine, or uracil). Modified molecules may be used, e.g. with modified backbones or non-naturally occurring nucleotides or naturally occurring modified nucleotides such as pseudouridine, 2-thiouridine, 5-methyluridine, 5-methylcytidine or N6-methyladenosine, so as to increase their half life providing this does not affect their functionality. Thus, the term “mRNA” thus also includes such modified molecules, i.e. encompasses derivatives or variants of mRNA which exhibit the same function, i.e. interaction with a ribosome and translation to express an encoded sequence. Preferred variants include those in which a modified backbone has been used (as above) or one or more non-naturally occurring bases or naturally occurring modified nucleotides (which may be introduced during synthesis) is used.

As is the case for DNA, RNA can form complementary hydrogen bonds, and RNA may be double-stranded (dsRNA), single-stranded (ssRNA) or double-stranded with a single-stranded overhang. Preferably the mRNA used in accordance with the invention is single stranded. The single stranded molecule may form tertiary structures which include double stranded regions, e.g. hairpin structures formed through internal base-pairing. Preferably the mRNA has a 5′ cap and a 3′ poly(A) tail (e.g. 120-150 nucleotides in length). Flanking untranslated regions may be present at the 3′ and/or 5′ end. Preferably, said mRNA molecules are 50-10,000 nucleotides long, more preferably 50-1000 or 1000-5000, e.g. 100-500 or 1500-2500 nucleotides long (when considering the sense strand).

The mRNA is naked. By this it is meant that it is not associated with a carrier or other molecule, i.e. is not bound or conjugated to or carried by any other component to aid its internalization. Solvents or solutions such as water, which solubilize or provide a diluent for the mRNA, are not considered to be carriers as they have no effect on the molecule's internalization. Such association includes any connection whether by binding, steric entrapment or other method that connects the molecules together so that they would remain associated under appropriate conditions. Thus no transfection agent or carrier is used. In this sense, the mRNA that is used is naked, i.e. free of associated molecules affecting its internalization. The photosensitising agent that is used with the mRNA in methods described herein does not constitute a carrier or transfection agent for the mRNA.

Preferably the mRNA encodes a polypeptide, i.e. carries sufficient consecutive coding codons that if translated would form a polypeptide. The polypeptide may include a signal peptide to allow processing and/or transport once translated. The polypeptide may comprise a single functional entity or contain multiple functional entities, e.g. the polypeptide may contain one or more peptide antigens, which may be used for vaccination. The mRNA may encode more than one polypeptide such that the result of translation is more than one polypeptide. Non-coding, e.g. stop codons, may also be present in the mRNA molecule. As referred to herein the “polypeptide” (which encompasses a peptide) comprises at least 5 consecutive amino acids. In a preferred aspect the polypeptide is at least 10, 20 or 30 amino acids in length, and less than 3000, 2000, 1000, 700, 500, 200 or 100 amino acids in length e.g. from 10-100, 200, 500, 700, 1000, 2000 or 3000 amino acids in length.

In a preferred aspect the polypeptide is expressed in the cell. The mRNA once internalized into the cell is bound by a ribosome and the mRNA translated into an amino acid sequence using the cell's gene expression machinery. The polypeptide is preferably a therapeutic molecule, i.e. a polypeptide with therapeutic properties such as a vaccine polypeptide, an antibody, an enzyme, a cytokine, a growth factor or a peptide hormone, for example.

The method may be used to introduce more than one type of mRNA molecule into a cell. In other words, mRNA molecules having different sequences can be introduced simultaneously into a cell. The molecules they express may act in different ways or interact with one another.

Appropriate methods for preparing mRNAs are known in the art and include chemical synthesis, in vitro transcription, mRNA expression vectors, and PCR expression cassettes. Such techniques are well known in the art. See for example Pon et al., 2005, Nucleosides Nucleotides Nucleic Acid. 24(5-7), 777-81, Du et al., 2006, Biochem. Biophys. Res. Commun. 345(1), 99-105 and Katoh et al., 2003, Nucleic Acids Res Suppl. (3), 249-50, Sahin et al., 2014, Nat. Rev. Drug Discov., 13(10), 759-780. mRNA for use in the invention may also be isolated from cells or tissues. In particular this allows personalized treatment, e.g. using mRNA isolated from the tumour of a subject to allow expression of patient specific tumour antigens in cancer immunotherapy.

The method of the invention achieves translocation of the mRNA molecule into the cytosol. It will be appreciated however that uptake of each and every molecule contacted with the cell is not achievable. Significant and improved uptake relative to background levels in which no PCI is used is however achievable.

Preferably methods of the invention allow the uptake of mRNA molecules at sufficient levels that their effect is evident in the expressed products of those cells. The appropriate concentration of mRNA to be contacted with the cell may be adjusted to achieve this aim, e.g. to achieve expression of the encoded sequence to desired levels after incubation with cells for e.g. 24, 48, 72 or 96 hours (e.g. 24 to 48 hours). The photosensitising agent type and/or concentration and the irradiation time can also be adjusted to achieve the expression required.

As used herein “and/or” refers to one or both (or more) of the recited options being present, e.g. A, B and/or C includes the options i) A, ii) B, iii) C, iv) A and B, v) A and C, vi) B and C, and vii) A, B and C.

Levels of expression can be measured by determining the level of protein in the cell, using standard techniques known in the art such as Western Blotting.

Expression may be assessed relative to expression achieved without the use of PCI. Comparisons can be made between the levels of protein expression that are seen at a certain mRNA (and/or photosensitiser) concentrations, in the presence and absence of PCI. For example, the method of the invention, including the therapeutic method, preferably allows enhanced expression of polypeptides of at least 10%, e.g. at least 20, 30, 40, 50, 60, 70, 80 or 90% or higher e.g. at least 100, 200, 300, 400 or 500%, compared to polypeptide expression achieved by carrying out the method in the absence of the irradiation step of the PCI technique. In a particularly preferred aspect, the method of the invention improves expression relative to delivery using lipofectamine without PCI by at least 10%, e.g. at least 20, 30, 40, 50, 60, 70, 80 or 90% or higher e.g. at least 100, 200, 300, 400 or 500%. Conveniently, the encoded polypeptide is expressed such that amounts of 1-100 μg are produced in the subject. Such amounts are sufficient for vaccination and immunotherapy. Larger amounts may generated, e.g. 1-100 mg when the polypeptide is used for direct therapeutic purposes, e.g. in protein therapy methods.

The “cell” or “cells” are in a body, namely a subject. The term “cell” or “cells” is used interchangeably herein. Thus the cell is provided within a subject or organism, i.e. an in vivo cell. The term “cell” include all eukaryotic cells (including insect cells). Representative “cells” thus include all types of mammalian and non-mammalian animal cells and insect cells. Preferably however the cells are mammalian, for example cells from monkeys, cats, dogs, horses, donkeys, sheep, pigs, goats, cows, mice, rats, rabbits, guinea pigs, but most preferably from humans. In the alternative the cells may be piscine cells.

The “subject” refers to a mammal, reptile, bird, insect or fish. Preferably the subject is a mammal, particularly a primate (preferably a human), domestic or companion animal, livestock or laboratory animal. In an alternative preferred aspect the subject is a fish. Thus preferred animals include mice, rats, rabbits, guinea pigs, cats, dogs, monkeys, pigs, cows, goats, sheep, donkeys, horses and fish.

As used herein “contacting” refers to bringing the cells and the photosensitising agent and/or mRNA into physical contact with one another under conditions appropriate for internalization into the cells. In methods and uses of the invention the contact occurs in vivo. Preferred methods of performing the invention including the timing and options for the contacting step are discussed hereinafter.

The duration of the contacting step refers to the time that the agents are in contact with the target cells to which they are administered before irradiation, i.e. the time from which both agents come into contact with the target cells until irradiation. (The target cells are those which are to be treated, e.g. cancer cells, and into which the mRNA is to be released.) Where the mRNA and photosensitiser are brought into contact with the cells simultaneously, the duration of the contacting step is from the start of that contact (at the same time) to irradiation. (When local, direct, administration to target cells is used, the start of contact commences at the same time as administration.) However, when both agents are not brought into contact with the cells simultaneously, the duration of the contacting step is the time both agents are in contact with the cells after administration until irradiation. Thus, for example if the mRNA is administered directly to target cells at time 0 and the photosensitiser is administered directly to target cells at 1 minute and irradiation is performed at 3 minutes, the contacting step (i.e. for both agents) is 2 minutes.

In sequential administration methods and uses, the timing between the start of contact of the mRNA and the start of contact of the photosensitiser (or vice versa) with the cells is preferably less than 5 minutes, preferably less than 60 seconds, especially preferably less than 30 seconds. In some such cases administration and contact with target cells may occur at the same time, i.e. when administration is directly to the target cells.

The photosensitising agent is an agent which is activated on illumination at an appropriate wavelength and intensity to generate an activated species. Conveniently such an agent may be one which localises to intracellular compartments, particularly endosomes or lysosomes. A range of such photosensitising agents are known in the art and are described in the literature, including in WO96/07432, which is incorporated herein by reference. In accordance with the invention, the photosensitising agent which is used is a sulphonated meso-tetraphenyl chlorin, sulfonated tetraphenylporphine or a di- or tetrasulfonated aluminium phthalocyanine.

In a preferred aspect the meso-tetraphenyl chlorin is TPCS₂ₐ (tetraphenyl chlorin disulfonate) or TPBS₂ₐ (tetraphenyl bacteriochlorin disulfonate), the sulfonated tetraphenylporphine is TPPSₙ, e.g. TPPS₄ or TPPS₂ₐ (tetraphenylporphine sulfonate or disulfonate), and the di- or tetrasulfonated aluminium phthalocyanine is AlPcS₂ₐ (aluminium phthalocyanine disulfonate). Pharmaceutically acceptable salts thereof may be used.

Particularly preferred are TPCS₂ₐ and TPPS₂ₐ, the structures of which are provided below.

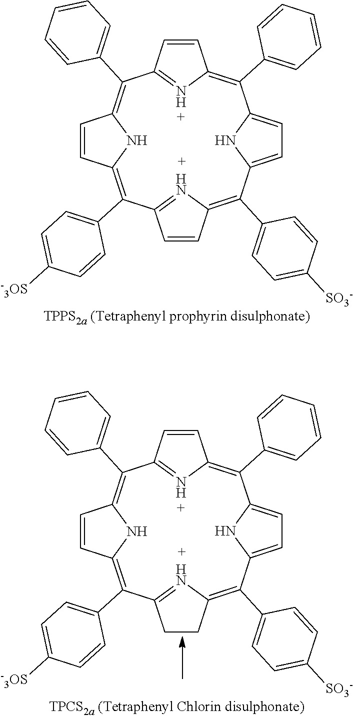

The arrow indicates the structural difference between the two molecules. Optionally, the photosensitising agent may be attached to or associated with or conjugated to one or more carrier molecules or targeting molecules which can act to facilitate or increase the uptake of the photosensitising agent. Thus the photosensitising agent may be linked to a carrier. For example, the photosensitising agent may be provided in the form of a conjugate, e.g. a chitosan-based conjugate, for example a conjugate disclosed in WO2013/189663, which is hereby incorporated by reference.

Whilst location specificity may be achieved by local delivery and activation by irradiation at the site of interest, if desired, the photosensitising agent may be targeted to specific cells (e.g. cancer cells) or tissues, by being associated or conjugated to specific targeting molecules that will promote the specific cellular uptake of the photosensitising agent molecule into desired cells or tissues.

Many different targeting molecules can be employed, e.g. as described in Curiel, 1999, Ann. New York Acad. Sci. 886, 158-171; Bilbao et al., 1998, in Gene Therapy of Cancer (Walden et al., eds., Plenum Press, New York); Peng and Russell, 1999, Curr. Opin. Biotechnol. 10, 454-457; Wickham, 2000, Gene Ther. 7, 110-114.

“Irradiation” of the cell to activate the photosensitising agent refers to the administration of light directly or indirectly as described hereinafter (and is sometimes referred to as illumination herein). Thus cells may be illuminated with a light source for example directly or indirectly, e.g. in vivo when the cells are below the surface of the skin or are in the form of a layer of cells not all of which are directly illuminated, i.e. without the screen of other cells. Preferred methods of irradiation are as described hereinafter.

Conveniently, the method may be carried out as now described. Where a carrier is to be used for the photosensitising agent, the carrier may be associated, bound or conjugated to the photosensitising agent by simply mixing the two components under appropriate conditions and concentrations and allowing the components to interact. The conditions under which this contacting step is carried out, and appropriate concentrations for each the carrier and the photosensitising agent can readily be determined by the person skilled in the art by carrying out routine testing. In the method of the invention, the mRNA molecule and the photosensitising agent (optionally with a carrier and/or a targeting molecule) are applied simultaneously, separately or sequentially to the cells, whereupon the photosensitising agent and the mRNA molecule are endocytosed or in other ways translocated into endosomes, lysosomes or other intracellular membrane restricted compartments or directly into the cell via the plasma membrane. As discussed below in some cases the photosensitising agent may not enter the cell but may allow transmission of the mRNA into the cell.

The mRNA molecule and the photosensitising agent may be applied to the cells together or sequentially. Conveniently the mRNA is administered to the cell simultaneously with the photosensitising agent (though they may be administered separately, e.g. sequentially). This is particularly important when a short contacting step before irradiation is to be used. The mRNA molecule and the photosensitising agent may be taken up by the cell into the same or different intracellular compartments (e.g. they may be co-translocated). Depending on the timing of irradiation after administration of the photosensitiser/mRNA, these molecules may or may not have been taken up by the cell at the time of irradiation. For example where very short duration contacting steps are used, some molecules may have been taken up but others may not, but may continue to be taken up by the cell after irradiation. In some cases the mRNA/photosensitiser may be taken up directly through the plasma membrane without being taken up into intracellular compartments, particularly once the photosensitiser has been activated which may have an effect on the integrity of the plasma membrane, as discussed below.

The exposure of the cells to light of suitable wavelengths activates the photosensitising agent which in turn leads to the disruption of the intracellular compartment membranes and potentially also the plasma membrane. In light of the amphiphilic nature of the photosensitiser, it may be associated with the plasma membrane and activated at that site leading to disruption of that membrane. Without wishing to be bound by theory, it is assumed that the disrupted plasma membrane may allow enhanced uptake of the mRNA which has not yet been taken up by the cells. The mRNA, which may be located in the same compartment as the photosensitising agent, may be released into the cytosol, or enter directly through the plasma membrane, on activation of the photosensitising agent. Thus, in these methods the final step of exposing the cells to light results in the mRNA entering the cytosol, by release from the same intracellular compartment as the photosensitising agent and/or via the plasma membrane.

“Internalisation” as used herein, refers to the cytosolic delivery of molecules. In the present case “internalisation” thus includes the step of release of molecules from intracellular/membrane bound compartments into the cytosol of the cells or entry of molecules into the cytosol via the plasma membrane.

As used herein, “cellular uptake” or “translocation” refers to one of the steps of internalisation in which molecules external to the cell membrane are taken into the cell such that they are found interior to the outerlying cell membrane, e.g. by endocytosis or other appropriate uptake mechanisms, for example into or associated with intracellular membrane-restricted compartments, for example the endoplasmic reticulum, Golgi body, lysosomes, endosomes etc, or simply by transfer across the plasma membrane.

The step of contacting the cells with a photosensitising agent and with the mRNA molecule may be carried out in any convenient or desired way. As discussed above, these agents may be applied to cells together, separately, simultaneously or sequentially.

The photosensitising agent is brought into contact with the cells at an appropriate concentration and for an appropriate length of time (subject to the restrictions discussed herein) which can easily be determined by a skilled person using routine techniques and will depend on such factors as the particular photosensitising agent used, the mode of administration, the target cell type and location, the course of treatment, the age and weight of the patient/subject, the medical indication, the body or body area to be treated and may be varied or adjusted according to choice. The concentration of the photosensitising agent must be such that once taken up into the cell, e.g. into, or associated with, one or more of its intracellular compartments and activated by irradiation, one or more cell structures are disrupted e.g. one or more intracellular compartments are lysed or disrupted, or such that it is capable of disruption of the plasma membrane.

The photosensitising agent is used in an amount of 0.000001 to 0.001 μg (or less than 0.001 μg), preferably 0.00001 to 0.0001 μg (or less than 0.0001 μg). Other preferred ranges include 0.000001 to 0.00001 μg, 0.000001 to 0.0001 μg (or less than 0.0001 μg), 0.000003-0.0005 μg, 0.000003 to 0.0001 μg, 0.000001-0.0003 μg, 0.000003-0.0003 μg or 0.0001-0.0003 μg. This is considerably lower than amounts used routinely for PCI such as 25 μg or the amounts used in WO2019/145419. The dose may be selected depending on the mode of administration and the light dose. For example, for intradermal or intramuscular administration a dose of 0.000001 to 0.001 μg may be selected (e.g. 0.000001 to 0.0001 μg, or less than 0.0001 μg). The same or higher doses may be used for intratumoural delivery, e.g. a dose of 0.00001 to 0.001 μg (e.g. 0.0001 to 0.0003 μg) may be selected. The above described dose may be used for local delivery to small local areas (less than a cubic cm). When larger areas are to be treated, the dose may be scaled accordingly.

The photosensitising agent is conveniently provided in a solution with a concentration of 0.0005 to 1 μg/ml, preferably 0.005 to 0.5 μg/ml. This concentration is suitable for local delivery.

Similar considerations apply to the mRNA. The mRNA is preferably used in an amount of 0.1 to 100 μg, e.g. 1 to 10 μg. The dose may be selected depending on the mode of administration as discussed above. The above described dose may be used for local delivery to small local areas (less than a cubic cm). When larger areas are to be treated, the dose may be scaled accordingly. The RNA is conveniently provided in a solution with a concentration of 5 to 5000 μg/ml, preferably 50 to 500 μg/ml. This concentration is suitable for local delivery.

An appropriate concentration should also take into account the mRNA molecule and photosensitising agent in question, the cells in question and the final concentration it is desired to achieve in the cells. As discussed herein, short contacting steps result in surprisingly high uptake of the molecule in question.

In accordance with the invention, the contacting step between the cell(s), the photosensitising agent and the mRNA is from 30 seconds to 10 minutes, preferably 30 seconds to 60 seconds (or 2, 3 or 4 minutes), or 30 seconds to 5 minutes. When administered simultaneously and directly to the target cells the duration of the contacting step is the same as the total time of incubation of the cell(s) with the photosensitising agent and the mRNA. However, when used sequentially, the photosensitising agent or the mRNA may be in contact with the cells for a time longer than the duration of the contacting step. Thus, for example if the contacting step is 2 minutes long, but the mRNA is added 1 minute before the photosensitising agent, the mRNA is incubated with the cells for a total of 3 minutes.

The mRNA molecule is brought into contact with the cells at an appropriate concentration and for an appropriate length of time. Appropriate concentrations are as discussed above. Contact times and the length of the contacting step are as discussed above for the photosensitising agent.

In preferred aspects, the mRNA and photosensitising agent are brought into contact with the cell(s) at the same time, which starts the contacting step, prior to irradiation.

Achieving an appropriate time of incubation (or contacting step duration) by which the mRNA molecule and photosensitising agent are brought into contact with the target cells in vivo will be dependent on factors such as the mode of administration and the type of mRNA molecule and photosensitising agent. For example, if the mRNA molecule is injected into a tumour, tissue or organ which is to be treated, the cells near the injection point will come into contact with and hence tend to take up the mRNA molecule more rapidly than the cells located at a greater distance from the injection point, which are likely to come into contact with the mRNA molecule at a later time point and lower concentration.

In light of the need to have a short, controlled, contacting step in which the cells and the mRNA and photosensitising agent are in contact, local administration of the agents is required.

For the avoidance of doubt, the time of the contacting step refers to the period of time in which the agents are both, together, in direct contact with the target cell(s). However, one of the agents may be in contact with the target cell(s) before this contacting step (requiring both agents) commences. Furthermore, the time of administration may precede the time of contact as the agents make their way to the target cells. When local administration is used direct contact begins immediately or shortly after administration.

The light irradiation step to activate the photosensitising agent may take place according to techniques and procedures well known in the art. The dose, wavelength and duration of the illumination must be sufficient to activate the photosensitising agent, i.e. to generate reactive species. Suitable light sources are well known in the art.

The wavelength of light to be used is selected according to the photosensitising agent to be used. Light of a wavelength effective to activate the photosensitising agent is able to elicit the production of reactive oxygen species on exposure of the photosensitising agent to that light. Suitable artificial light sources are well known in the art, e.g. using blue (400-475 nm) or red (620-750 nm) wavelength light. For TPCS₂ₐ, for example, a wavelength of between 400 and 500 nm, more preferably between 400 and 450 nm, e.g. from 400-435 nm or 420-435 nm, and even more preferably approximately 435 nm, or 435 nm may be used. In the alternative, red light may be used to ensure deeper light penetration, e.g. for tumour tissue. In this case a wavelength of 620-750, e.g. 640-660 nm may be used. Where appropriate the photosensitiser, e.g. a porphyrin or chlorin, may be activated by green light, for example the KillerRed (Evrogen, Moscow, Russia) photosensitiser may be activated by green light.

Suitable light sources are well known in the art, for example the LumiSource® lamp of PCI Biotech AS. Alternatively, an LED-based illumination device which has an adjustable output power of up to 60 mW and an emission spectra of 400-435 nm may be used. For red light, a suitable source of illumination is the PCI Biotech AS 652 nm laser system SN576003 diode laser, although any suitable red light source may be used.

Appropriate light doses can be selected by a person skilled in the art and again will depend on the photosensitising agent and the amount of photosensitising agent accumulated in the target cells or tissues. For example, the light dose typically used for photodynamic treatment of cancers with the photosensitiser Photofrin and the protoporphyrin precursor 5-aminolevulinic acid is in the range 50-150 J/cm² at a fluence range of less than 200 mW/cm² in order to avoid hyperthermia. The light doses are usually lower when photosensitising agents with higher extinction coefficients in the red area of the visible spectrum are used. However, for treatment of non-cancerous tissues with less photosensitiser accumulated, the total amount of light needed may be substantially higher than for treatment of cancers. Furthermore, if cell viability is to be maintained, the generation of excessive levels of toxic species is to be avoided and the relevant parameters may be adjusted accordingly.

Appropriate light doses can be selected by a person skilled in the art and again will depend on the photosensitising agent used and the amount of photosensitising agent accumulated in the target cell(s) or tissues. The light doses are usually lower when photosensitisers with higher extinction coefficients (e.g. in the red area, or blue area if blue light is used, depending on the photosensitiser used) of the visible spectrum are used. For example, a light dose in the range of 0.24-7.2 J/cm² at a fluence range of 0.05-20 mW/cm², e.g. 2.0 mW/cm², may be used when an LED-based illumination device which has an adjustable output power of up to 60 mW and an emission spectra of 400-435 nm or 420-435 nm is employed. Alternatively, e.g. if the LumiSource® lamp is employed a light dose in the range of 0.1-6 J/cm² at a fluence range of 0.1-20 (e.g. 13 as provided by Lumisource®) mW/cm² is appropriate. For red light, a light dose of 0.03-8 J/cm², e.g. 0.03-4 J/cm², e.g. 0.3 J/cm², at a fluence range of 0.1-5 mW/cm², e.g. 0.81 mW/cm², may be used.

Conveniently, the light source and irradiation time is selected such that the cell(s) is irradiated with a light dose of from 0.01 to 50 J/cm², such as 0.1 to 10 J/cm², e.g. 0.4 to 5 J/cm² with a 13 mW/cm² fluence rate. Preferably the cell(s) is irradiated with a light dose of from 0.3 to 3 J/cm² or 0.3 to 6 J/cm².

The time for which the cells are exposed to light in the methods of the present invention may vary. The efficiency of the internalisation of the mRNA molecule into the cytosol generally increases with increased exposure to light to a maximum beyond which cell damage and hence cell death increases.

A preferred length of time for the irradiation step depends on factors such as the target, the photosensitising agent, the amount of the photosensitising agent accumulated in the target cells or tissue and the overlap between the absorption spectrum of the photosensitising agent and the emission spectrum of the light source. Generally, the length of time for the irradiation step is in the order of minutes to hours, e.g. preferably up to 60 minutes e.g. from 15 seconds to 60 minutes, preferably from 0.5 to 12 minutes, preferably for 4 to 6 minutes.

The methods of the invention may inevitably give rise to some cell killing by virtue of the photochemical treatment i.e. through the generation of toxic species on activation of the photosensitising agent. Depending on the proposed use, this cell death may not be of consequence and may indeed be advantageous for some applications (e.g. cancer treatment). Preferably, however, cell death is avoided to allow translation of the mRNA and expression of the encoded polypeptide. The methods of the invention may be modified such that the fraction or proportion of the surviving cells is regulated by selecting the light dose in relation to the concentration of the photosensitising agent. Again, such techniques are known in the art.

In applications in which viable cells are desirable, substantially all of the cells, or a significant majority (e.g. at least 50%, more preferably at least 60, 70, 80 or 90% of the cells) are not killed. Cell viability following PCI treatment can be measured by standard techniques known in the art such as the MTS test.

Regardless of the amount of cell death induced by the activation of the photosensitiser, for the mRNA molecule to have an effect in the cells, it is important that the light dose is regulated such that some of the individual cells wherein the PCI effect is manifested are not killed by the photochemical treatment alone (although they may subsequently be killed by molecules introduced into the cells if those molecules have a cytotoxic effect).

Cytotoxic effects may be achieved by using, for example, an mRNA molecule which is internalized into a tumour cell by the method of the invention and which by expresses a cytotoxic molecule.

The methods of the invention are used in vivo for various purposes including expression of specific gene products e.g. in protein therapy, immunotherapy and gene therapy methods.

Thus the present invention provides an in vivo method of expressing a polypeptide in a cell(s) in a subject by introducing an mRNA molecule into a cell(s) by a method as defined hereinbefore, wherein said mRNA molecule encodes said polypeptide.

These methods may be used to alter the expression profile of cells or to determine the influence of expression of a particular gene, and/or for therapeutic purposes.

The methods of the invention may also be used in treating any disease, disorder or infection which benefits from expression of a polypeptide, e.g. by expression of one or more genes to provide therapeutic molecules which act directly or indirectly. Such molecules may act directly to provide a therapeutic result or may act indirectly, e.g. by generating an immune response or aiding alteration of gene expression and thus provide gene therapy to a subject, as discussed in more detail hereinafter. Such therapeutic molecules include therapeutic antibodies (or antigen-binding fragments thereof) that may be targeted to appropriate sites to treat diseases, infections or disorders. The therapeutic molecule may also be an enzyme or other functional molecule required for metabolism, e.g. growth factors, cytokine or peptide hormones. Alternatively, an inhibitor or cell death inducing molecule may be used, e.g. a cytotoxic molecule. Conveniently, the expressed polypeptide may provide an antigenic molecule against which an immune response may be generated, e.g. for prophylactic or therapeutic vaccination. The immune response may be generated against pathogenic infections, e.g. bacterial or viral infections or against aberrant cells in the body, e.g. cancer cells. Thus the polypeptide may be an antigenic molecule such as a cancer vaccine or a bacterial or viral antigen. Preferred uses of the invention are discussed in more detail hereinbelow.

The invention provides compositions suitable for the therapeutic uses. Thus, the invention provides a pharmaceutical composition comprising an mRNA molecule and a photosensitising agent, wherein said mRNA is naked and said photosensitising agent is a sulphonated meso-tetraphenyl chlorin, sulfonated tetraphenylporphine or a di- or tetrasulfonated aluminium phthalocyanine and is provided in the amount of 0.000001 to less than 0.0001 μg. Also provided is the composition for use in therapy. Preferably said photosensitising agent and/or said mRNA is as defined hereinbefore. A pharmaceutical composition comprises in addition to the active ingredient(s) one or more pharmaceutically acceptable diluents, carriers or excipients.

Alternatively, the mRNA and photosensitising agent may be in separate solutions or compositions allowing different mechanisms or timings for administration or application. As referred to herein “co-administration” and “co-application” refers to use of both components in the same method rather than simultaneous use (either in terms of timing or in the same composition).

Alternatively, the present invention provides a kit comprising an mRNA molecule and a photosensitising agent as described herein. Preferably said kit (or product) is for simultaneous, separate or sequential use in a medical treatment.

The invention further provides an mRNA molecule and a photosensitising agent for use in treating or preventing a disease, disorder or infection in a subject by expressing a polypeptide encoded by said mRNA molecule, wherein said mRNA is naked and said photosensitising agent is a sulphonated meso-tetraphenyl chlorin, sulfonated tetraphenylporphine or a di- or tetrasulfonated aluminium phthalocyanine used in an amount of 0.000001 to 0.001 μg, and one or more cells in said subject are to be contacted with said mRNA molecule and photosensitising agent and irradiated with light of a wavelength effective to activate the photosensitising agent and said contacting step is to be performed for 30 seconds to 10 minutes. Preferably the photosensitising agent and/or said mRNA is as defined hereinbefore and the intended treatment or prevention is preferably carried out using a method as described hereinbefore.

In an alternative description of the invention, the present invention provides the use of an mRNA molecule and a photosensitising agent in the preparation of a medicament for treating or preventing a disease, disorder or infection in a subject by expressing a polypeptide encoded by said mRNA molecule, wherein said mRNA is naked and said photosensitising agent is a sulphonated meso-tetraphenyl chlorin, sulfonated tetraphenylporphine or a di- or tetrasulfonated aluminium phthalocyanine used in an amount of 0.000001 to 0.001 μg and one or more cells in said subject are to be contacted with said mRNA molecule and photosensitising agent and irradiated with light of a wavelength effective to activate the photosensitising agent and said contacting step is to be performed for 30 seconds to 10 minutes. Preferably said photosensitising agent and/or said mRNA are as defined herein. Preferably said cells are subject to a method as described herein.

Optionally said medicament may contain only one of said mRNA molecule and photosensitising agent and may be used in methods in which said mRNA molecule or photosensitising agent which is not present in said medicament is for administration to said patient (or subject) when treating or preventing said disease, disorder or infection.

In a further alternative description of the invention, the present invention provides a method of treating or preventing a disease, disorder or infection in a subject comprising introducing an mRNA molecule into one or more cells in vivo in said subject according to the methods as defined herein.

As defined herein “treatment” refers to reducing, alleviating or eliminating one or more symptoms of the disease, disorder or infection which is being treated, relative to the symptoms prior to treatment. “Prevention” (or preventing or prophylaxis) refers to delaying or preventing the onset of the symptoms of the disease, disorder or infection. Prevention may be absolute or may be effective only in some individuals, or cells, or for a limited amount of time.

The disease, disorder or infection to be treated or prevented may be any condition which would benefit from the expression of one or more polypeptides. Such conditions may exhibit low or no expression of the polypeptide, e.g. when the endogenous polypeptide is not expressed at required levels, is absent or higher levels would be therapeutic (e.g. to correct metabolic processes or for vaccination), or would benefit from use of an exogenous polypeptide for therapeutic purposes, e.g. for vaccination or to achieve cell death, e.g. a cytotoxic molecule. In another aspect, and as described hereinafter, the therapy may be gene therapy. In some instances the gene therapy may provide a gene to replace a defective or missing equivalent in the subject. The expressed polypeptides may act directly in a therapeutic manner (e.g. a cytotoxic molecule) or may initiate a therapeutic response, e.g. a therapeutic immune response. Particularly preferred diseases, disorders or infections to be treated include cancer, cardiovascular disease, autoimmune diseases, cystic fibrosis, neurodegenerative diseases such as Huntington's disease, Alzheimer's disease and Parkinson's disease, viral infections such as influenza, hepatitis (e.g. B and C), HIV and herpes, infections with intracellular or extracellular bacteria, such as in tuberculosis, leprosy, chlamydia, listeria, legionella and cholera and infection by E. coli, P. aeruginosa, S. aureus, Streptococcus spp., N. meningitidis and S. pyogenes, infections by parasites, e.g. in malaria and leishmaniosis and other diseases, disorders or infections discussed herein.

The in vivo uses may be divided into protein therapy, immunotherapy and gene therapy methods.

In protein therapy methods, the mRNA is used to produce a protein that the patient is lacking, e.g. because of an inherited mutation or reduced expression, or which would have a therapeutic effect. In one alternative, this could be e.g. an enzyme, a peptide hormone, a cytokine, a growth factor, a blood clotting factor (in bleeding disorders) or other important proteins. In this case an mRNA encoding the missing protein is delivered to suitable cells in the body (for example in the skin, muscle, liver etc.) so that these cells produce the missing protein that will either act inside the producer cell (e.g. if the mRNA encodes an intracellular enzyme), locally (e.g. to produce a growth factor in a certain tissue) or systemically (e.g. to produce a missing blood clotting factor or a peptide hormone).

In a second alternative, the protein may be a protein which has a therapeutic effect, but is not necessarily naturally occurring. For example, the mRNA may encode one or more antibodies to an infectious agent. In this case, upon delivery of the mRNA to the body (into any tissue capable of producing the antibody protein and secreting it into the blood stream) the body will rapidly (4-6 hours) synthesize antibodies to the infectious agent that may rapidly stop the development of the infection. Such therapy will be very specific to the specific infectious agent, and will not be subject to problems e.g. with antibiotic resistance. Preferably this method may be used to treat acute infections (until the body's immune system can take over) resulting from pathogens, e.g. viruses, bacteria (particularly extracellular but also intracellular bacteria) and parasites. The method may also be used to supplement the body's immune response to treat various diseases, disorders or infections, including viral diseases such as influenza, hepatitis (e.g. B and C), HIV and Herpes and many other viral infections; infections with bacteria (extracellular or intracellular), such as in tuberculosis, leprosy, chlamydia, listeria, legionella and cholera and infection by E. coli, P. aeruginosa, S. aureus, Streptococcus spp., N. meningitidis and S. pyogenes and several other infections; infections by parasites, e.g. in malaria and leishmaniosis.

The antibodies may also be used for treatments in which antibody proteins are known to be useful. Such treatments include cancer, but also other disease groups, such as autoimmune diseases (rheumatoid arthritis, inflammatory bowel disease, etc.).

Other non-naturally occurring therapeutic proteins include cytotoxic molecules, e.g. to treat cancer.

In a third alternative, the mRNA may be used for regenerative purposes, e.g. to induce the local production of proteins that will help remodeling the target tissue in a desired way. Thus, for example, mRNA that encode factors that will promote proper healing in a wound (e.g. growth factors), or factors that will induce the formation of new blood vessels in ischemic tissues such as in the heart after an infarction, may be used. Another example is the use of mRNA to generate a pulse production of a paracrine factor to direct e.g. progenitor cells to differentiate in a way useful for generating a beneficial response, e.g. the regeneration of heart muscle and vessels after a heart infarction (Zangi et al., 2013, Nat. Biotechnol., 31 (10), 898-907). Similar principles may be used e.g. for repair of damage to neural tissues (e.g. due to physical damage, brain thrombosis or in Alzheimer's or Parkinson's disease), for regeneration of tissues in the eye and in many other types of tissue damage.

In a particularly preferred embodiment, the disease to be treated is cancer. In this case protein therapy may be achieved in a number of ways (immunotherapy may also or alternatively be used as described hereinafter). For example, mRNA can be used for the local or systemic expression of proteins that modulate anti-tumour immune responses, such as checkpoint inhibitors (e.g. monoclonal antibodies encoded by mRNAs), ligands that will activate co-stimulatory molecules on immune cells (e.g. a CD40 ligand), or factors acting in a paracrine fashion to modulate the tumour microenvironment in a way that may enhance anti-tumour immune responses, e.g. by acting on tumour-infiltrating macrophages.

In immunotherapy methods the expressed polypeptide is used to generate a therapeutic immune response. This may include prophylactic or therapeutic vaccination methods. Such methods may be used to treat infectious diseases. For example, prophylactic vaccination may be used in which a relevant antigen is used prior to exposure to the infectious agent to generate adaptive immunity to subsequent exposure. Preferred target infectious diseases are typically diseases in which T-cell responses are important. Examples include: viral diseases such as influenza, hepatitis (e.g. B and C), HIV, Herpes and many other viral infections; infections with bacteria (intracellular or extracellular), such as in tuberculosis, leprosy, chlamydia, listeria, legionella and cholera and infection by E. coli, P. aeruginosa, S. aureus, Streptococcus spp., N. meningitidis and S. pyogenes, and several other infections; infections by parasites, e.g. in malaria and leishmaniosis. Appropriate antigens are selected to generate a prophylactic immune response and the encoding mRNA used in methods of the invention.

Therapeutic vaccination is also contemplated, i.e. treatment of infected subjects by generation of an immune response to antigen expressed after mRNA internalization. In this case, preferred target diseases are chronic infections by viruses, bacteria (usually intracellular but also extracellular) and parasites, such as those described above for prophylactic vaccination.

Of particular interest is the use of immunotherapy in treating cancer. This includes both prophylactic and therapeutic vaccination, as described hereinbefore.

Gene therapy methods may also be used. As referred to herein gene therapy methods are considered to be methods which introduce or modify one or more gene within a subject or modify the expression of one or more gene in a subject. Thus, by way of example, the mRNA may encode a polypeptide that would assist in altering the subject's genome. Thus, for example, mRNA which encodes enzymes useful in sequence-specific modification of the chromosomal DNA in the target cells may be used, e.g. to correct a mutated gene or to insert a copy of a non-mutated version of a disease-causing mutated gene. Examples of such enzymes include Cas9 (CRISPR technology), zinc finger nucleases, transcription activator-like effector nuclease mRNA (TALEN mRNA) and site-specific recombinases. As necessary, in some cases the mRNA would be used together with a “donor DNA” e.g. to insert the “correct” DNA sequence to correct a mutation, in other cases the mRNA may be used alone, e.g. for inactivating a gene. In preferred aspects, the method may be used to treat Huntingdon's disease, cystic fibrosis and other inherited diseases.

As discussed above, in a particularly preferred aspect the method is used to generate an immune response, particularly to achieve vaccination. As referred to herein an immune response is any reaction of the host defence system in vivo. As referred to herein, “vaccination” is the use of an antigen (or a molecule containing an antigen) to elicit an immune response which is prophylactic or therapeutic against the development (or further development) of a disease, disorder or infection, wherein that disease, disorder or infection is associated with abnormal expression or presence of that antigen. Preferably the disease is cancer. In one embodiment the vaccination is therapeutic, for example in the treatment of cancers or chronic parasitic, bacterial or viral infections as described herein. In an alternative embodiment the vaccination is prophylactic, for example to prevent a cancer or to reduce further cancers developing following treatment of an earlier cancer with a therapeutic vaccination. In a further embodiment when an immune response to an infection is to be generated, e.g. an infection as discussed hereinbefore, e.g. a viral infection such as hepatitis or HIV infection, parasitic infections like malaria, or bacterial infections (e.g. tuberculosis), the vaccination is prophylactic in nature.

In methods of vaccination, the mRNA expresses a suitable antigenic molecule for vaccination purposes.

Many such antigens or antigenic vaccine components are known in the art and include all manner of bacterial or viral antigens or indeed antigens or antigenic components of any pathogenic species including protozoa or higher organisms. Whilst traditionally the antigenic components of vaccines have comprised whole organisms (whether live, dead or attenuated) i.e. whole cell vaccines, in addition sub-unit vaccines, i.e. vaccines based on particular antigenic components of organisms e.g. proteins or peptides, or even carbohydrates, have been widely investigated and reported in the literature. Any such “sub-unit”-based vaccine component may be used as the expressed polypeptide of the present invention.

However, the invention finds particular utility in the field of peptide vaccines, e.g. peptides of 5-500 e.g. 10 to 250 such as 15 to 75, or 8 to 25 amino acids.

A vast number of peptide vaccine candidates have been proposed in the literature, for example in the treatment of viral diseases and infections such as AIDS/HIV infection or influenza, canine parvovirus, bovine leukaemia virus, hepatitis, etc. (see e.g. Phanuphak et al., 1997, Asian Pac. J. Allergy. Immunol., 15(1), 41-8; Naruse, 1994, Hokkaido Igaku Zasshi, 69(4), 811-20; Casal et al., 1995, J. Virol., 69(11), 7274-7; Belyakov et al., 1998, Proc. Natl. Acad. Sci. USA, 95(4), 1709-14; Naruse et al., 1994, Proc. Natl. Sci. USA, 91(20), 9588-92; Kabeya et al., 1996, Vaccine, 14(12), 1118-22; Itoh et al., 1986, Proc. Natl. Acad. Sci. USA, 83(23) 9174-8. Similarly bacterial peptides may be used, as indeed may peptide antigens derived from other organisms or species.

In addition to antigens derived from pathogenic organisms, peptides have also been proposed for use as vaccines against cancer or other diseases such as multiple sclerosis. For example, mutant oncogene peptides hold great promise as cancer vaccines acting as antigens in the stimulation of cytotoxic T-lymphocytes. (Schirrmacher, 1995, Journal of Cancer Research and Clinical Oncology, 121, 443-451; Curtis, 1997, Cancer Chemotherapy and Biological Response Modifiers, 17, 316-327). A synthetic peptide vaccine has also been evaluated for the treatment of metastatic melanoma (Rosenberg et al., 1998, Nat. Med., 4(3), 321-7), and personalized mRNA-based vaccines based on peptide epitopes mutated in tumours of individual patients have shown great promise for cancer treatment (Sahin et al., 2017, Nature, 257, 222-226). A T-cell receptor peptide vaccine for the treatment of multiple sclerosis is described in Wilson et al., 1997, J. Neuroimmunol., 76(1-2), 15-28. Any such peptide vaccine component may be used as the expressed polypeptide according to the invention, as indeed may any of the peptides described or proposed as peptide vaccines in the literature. The mRNA used for vaccination may code for a single peptide antigen, or it may encode several different peptide antigens translated into one polypeptide, e.g. as described in Sahin et al., 2017, supra.

For administration of agents or cells described herein in vivo, any mode of administration common or standard in the art may be used, e.g. oral, parenteral (e.g. intramuscular, transdermal, subcutaneous, percutaneous, intraperitoneal, intrathecal or intravenous), intestinal, buccal, rectal or topical (i.e. direct application), both to internal and external body surfaces etc. However, particularly in light of the short contacting step, the administration is local, regardless of the mode of administration. The invention can be used in relation to any tissue which contains cells to which the photosensitising agent or the mRNA molecule (or cells containing the same) may be localized, including body fluid locations, as well as solid tissues. All tissues can be treated as long as the photosensitiser is taken up by the target cells, and the light can be properly delivered. When cells are to be administered methods are not constrained by the ability to deliver light. Preferred modes of administration are intradermal, intratumoural, subcutaneous, intramuscular, which may be achieved by topical administration or injection, particularly intradermal, intramuscular or intratumoural administration. Preferably administration is by injection. In this way the required agents are delivered directly to the target cells.

Thus, the compositions of the invention may be formulated in any convenient manner according to techniques and procedures known in the pharmaceutical art, e.g. using one or more pharmaceutically acceptable carrier or excipients. “Pharmaceutically acceptable” as referred to herein refers to ingredients that are compatible with other ingredients of the compositions as well as physiologically acceptable to the recipient. The nature of the composition and carriers or excipient materials, dosages etc. may be selected in routine manner according to choice and the desired route of administration, purpose of treatment etc. Dosages may likewise be determined in routine manner and may depend upon the nature of the molecule, purpose of treatment, age of patient, mode of administration etc. In connection with the photosensitising agent the potency/ability to disrupt membranes on irradiation, should also be taken into account.

To achieve the desired outcome, i.e. treatment or prevention of disease, disorder or infection, the methods or parts thereof may be repeated. Thus, the method in its entirety may be performed multiple times (e.g. 2, 3 or more times) after an appropriate interval or parts of the method may be repeated, e.g. further administration of the mRNA and/or photosensitising agent as defined herein or additional irradiation steps. For example, the method or part of the method may be performed again a matter of days, e.g. between 5 and 60 days (for example 7, 14, 15, 21, 22, 42 or 51 days), e.g. 7 to 20 days, preferably 14 days, or weeks, e.g. between 1 and 5 weeks (for example, 1, 2, 3 or 4 weeks) after it was first performed. All or part of the method may be repeated multiple times at appropriate intervals of time, e.g. every two weeks or 14 days. In a preferred embodiment the method is repeated at least once. In another embodiment the method is repeated twice.

The invention will now be described in more detail in the following non-limiting Examples with reference to the following drawings in which:

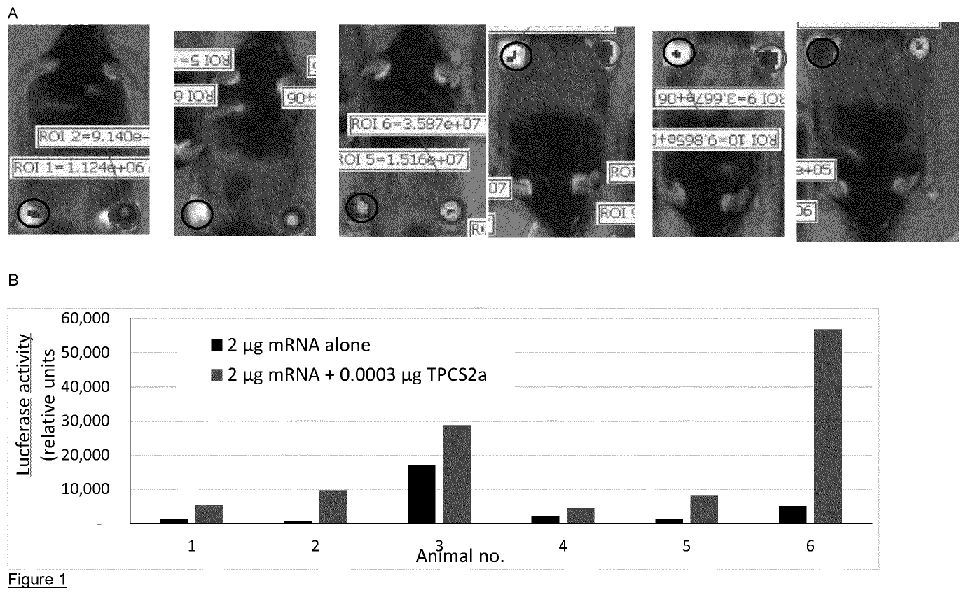

FIG. 1 shows (A) bio-luminescence imaging for the two injection sites in the thigh muscle of each mouse injected with a mixture of 2 μg luciferase mRNA (TriLink L-6107, 5 meC, ψ)) alone or with 0.003 μg TPCS₂ₐ (see Example 1, Table 1 for protocol) and illuminated with red light 5 minutes after administration, and (B) shows the luciferase activity in muscle tissue from those sites for each animal.

FIG. 2 shows the mean luciferase activity in the skin from the intradermal injection site of mice after injection of 2 μg luciferase mRNA alone or with 0.003 μg TPCS₂ₐ and illuminated with blue light at various times after administration, as indicated.

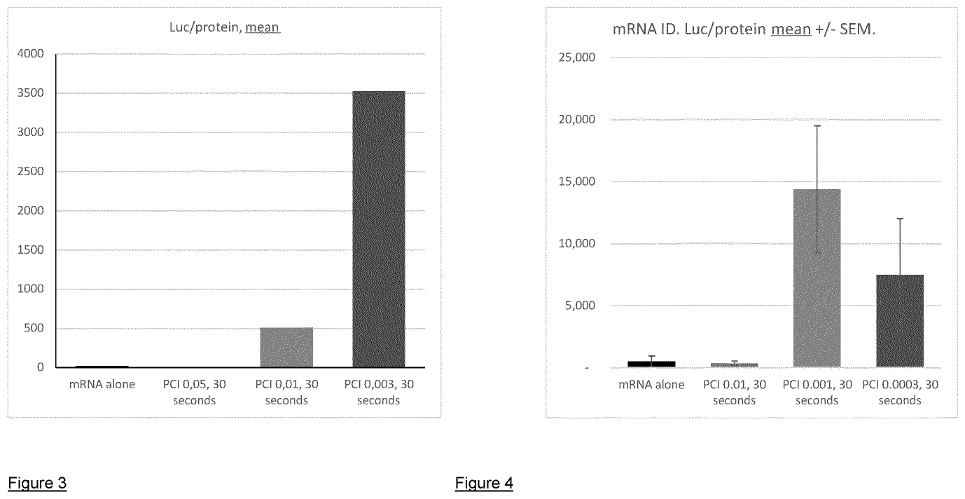

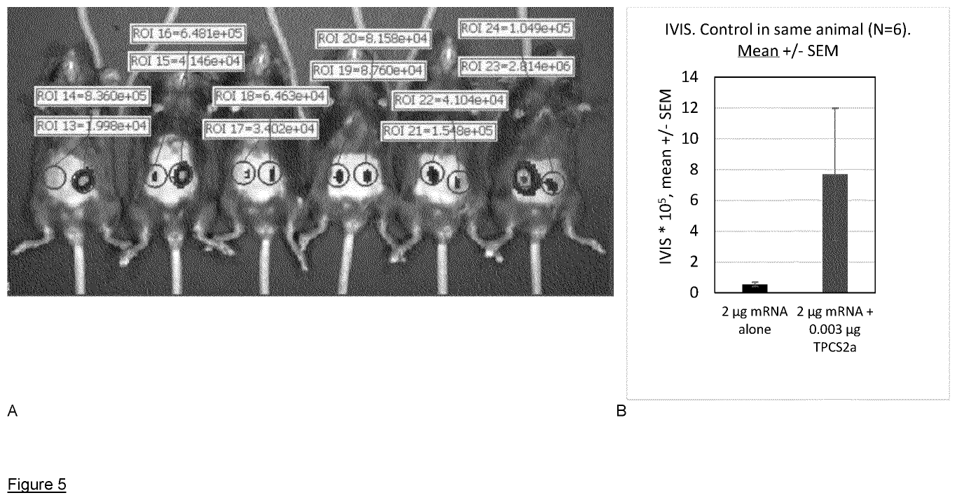

FIGS. 3 and 4 show the mean luciferase activity in the skin from the intradermal injection site of mice after injection of 2 μg luciferase mRNA alone or with various concentrations of TPCS₂ₐ (as indicated) and illuminated with blue light 30 seconds after administration.

FIG. 5 shows (A) bio-luminescence imaging for the two injection sites in the skin of each mouse after injection of 2 μg luciferase mRNA alone or with 0.003 μg TPCS₂ₐ and illuminated with blue light 30 seconds after administration and (B) the mean results from A.

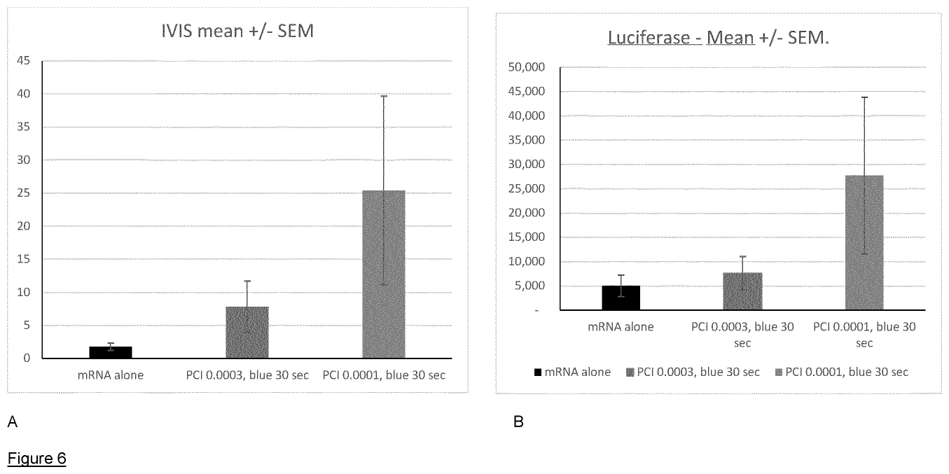

FIG. 6 shows (A) mean bio-luminescence at the injection site in the muscle for each group injected with 2 μg luciferase mRNA alone or with 0.0003 or 0.0001 μg TPCS₂ₐ and illuminated with blue light 30 seconds after administration, and (B) the mean luciferase activity in muscle tissue from those sites for each group.

FIG. 7 shows the mean luciferase activity in muscle tissue from the injection site of mice after injection of 2 μg luciferase mRNA alone or with various concentrations of TPCS₂ₐ (as indicated) and illuminated with blue light 30 seconds after administration.

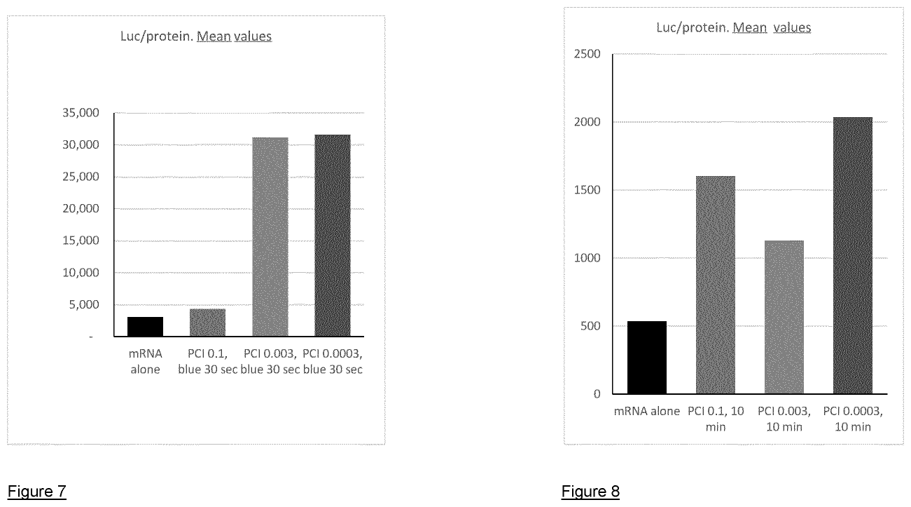

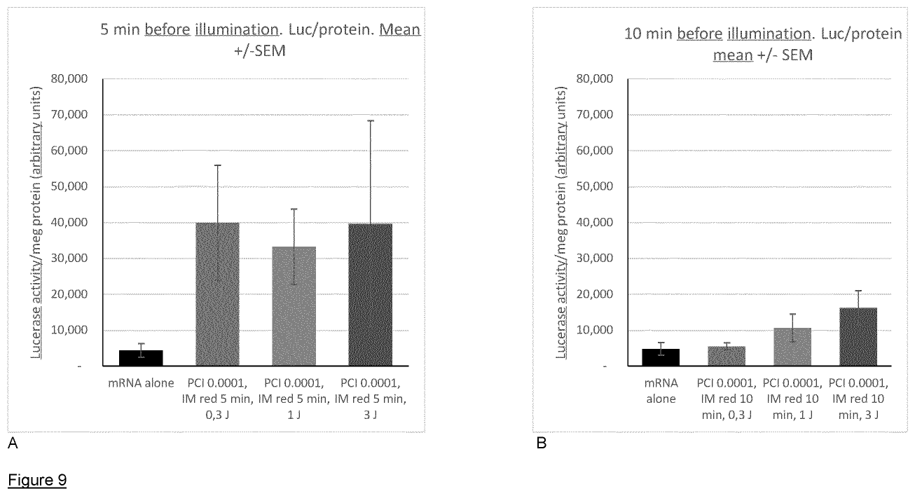

FIG. 8 shows the mean luciferase activity in muscle tissue from the injection site of mice after injection of 2 μg luciferase mRNA alone or with various concentrations of TPCS₂ₐ (as indicated) and illuminated with blue light 10 minutes after administration.

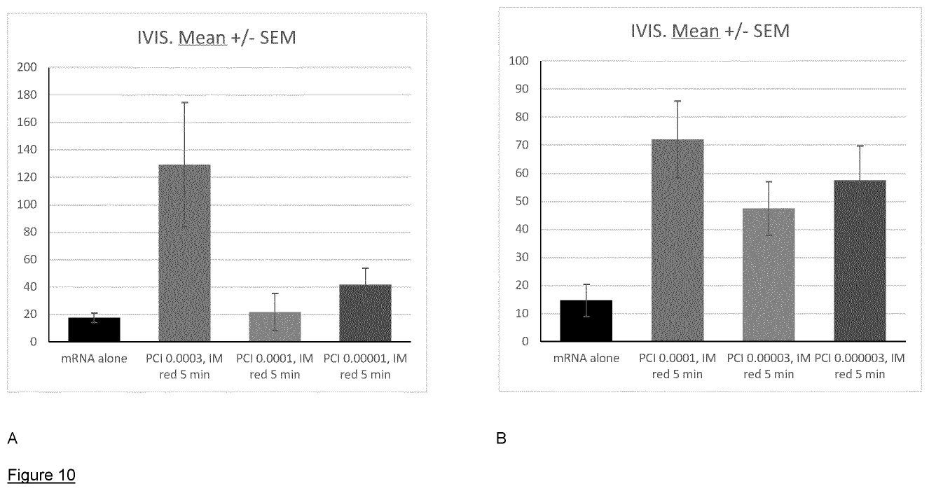

FIG. 9 shows the mean luciferase activity in muscle tissue from the injection site of mice after injection of 2 μg luciferase mRNA alone or with 0.0001 μg TPCS₂ₐ and illuminated with red light (at the doses indicated) 5 (FIG. 9A) or 10 minutes (FIG. 9B) after administration.

FIG. 10 shows the mean results of bio-luminescence imaging for the two intramuscular injection sites for the animals in each group for experiments 1 and 2 (FIGS. 10A and B, respectively), after injection of 2 μg luciferase mRNA alone or various concentrations of TPCS₂ₐ (as indicated) and illuminated with red light 5 minutes after administration.

### EXAMPLES

### Example 1: Intramuscular mRNA Delivery to BL/6 Mice In Vivo with Red Light Illumination 5 Minutes after mRNA/Photosensitiser Injection

Experiments were performed to study in vivo naked mRNA intramuscular delivery to mice using shorter contact times and lower photosensitiser doses than previously used.

### Materials and Methods

### Mice

Female mice of the strain C57BL/6 (Charles River) were used. Animal identification and conditions of housing, acclimatisation, environment, diet and water was in accordance with the current Standard Operating Procedures at the animal facilities at Oslo University Hospital—The Radium Hospital. Age and weight at start of dosing: 5-6 weeks, 18-20 g. The injection area was shaved before illumination.

mRNA

Firefly luciferase mRNA (L-7202—with modified base 5-methoxyuridine (5 moU), and with the TriLink CleanCap™ modification as the capping structure, purchased from TriLink Biotechnologies, San Diego, USA) was used.

### Photosensitiser

TPCS₂ₐ (Amphinex®, PCI Biotech AS, Norway) was used mixed in a volume of 20 μl PBS (e.g. using a 0.000015 μg/μl solution for a 0.0003 μg dose).

### Methods

mRNA (in aqueous solution, 20 μl of 0.1 μg/μl from stock solution of 1 μg/μl) in the amounts indicated in the table below were injected into the thigh muscle of each animal, with or without TPCS₂ₐ in the amounts indicated.

TABLE 1
Injection protocols

| Animal | Injection | Injection |
|---|---|---|
| no. | site A (left) | site B (right) |
| 1 | 2 μg mRNA alone | 2 μg mRNA + 0.0003 μg |
| | | TPCS₂ₐ |
| 2 | 2 μg mRNA alone | 2 μg mRNA + 0.0003 μg |
| | | TPCS₂ₐ |
| 3 | 2 μg mRNA alone | 2 μg mRNA + 0.0003 μg |
| | | TPCS₂ₐ |
| 4 | 2 μg mRNA + 0.0003 μg | 2 μg mRNA alone |
| | TPCS₂ₐ | |
| 5 | 2 μg mRNA + 0.0003 μg | 2 μg mRNA alone |
| | TPCS₂ₐ | |
| 6 | 2 μg mRNA + 0.0003 μg | 2 μg mRNA alone |
| | TPCS₂ₐ | |
| 7 | 2 μg mRNA alone, | 2 μg mRNA + 0.0003 μg |
| | no illumination | TPCS₂ₐ, no illum. |
| 8 | 2 μg mRNA alone, | 2 μg mRNA + 0.0003 μg |
| | no illumination | TPCS₂ₐ, no illum. |
| 9 | 2 μg mRNA alone, | 2 μg mRNA + 0.0003 μg |
| | no illumination | TPCS₂ₐ, no illum. |
| 10 | 2 μg mRNA + 0.0003 μg | 2 μg mRNA alone, |
| | TPCS₂ₐ, no illum. | no illumination |
| 11 | 2 μg mRNA + 0.0003 μg | 2 μg mRNA alone, |
| | TPCS₂ₐ, no illum. | no illumination |
| 12 | 2 μg mRNA + 0.0003 μg | 2 μg mRNA alone, |
| | TPCS₂ₐ, no illum. | no illumination |

In animals 1-6 both thigh muscles in each animal (“injection site right” and “injection site left”) were illuminated with red light for 6 minutes (using a laser emitting light with a wavelength peak of 652 nm, 1 J/cm²) 5 minutes after mRNA/TPCS₂ₐ injection. Animals 7-12 were not illuminated. 20 hours after the injections the animals were analysed by IVIS and assayed for luciferase activity (Luciferase Assay System, Promega, Cat #E1500) in muscle homogenates.

Muscle homogenates were prepared from frozen tissue samples using a Precelly homogenizer and lysis buffer with phosphatase and protease inhibitors (MSD, Cat. No. R60TX-2, R70AA-1). Protein analysis was performed using the DC Protein Assay (Biorad). Luciferase is expressed as relative units (RLU=relative luminescence units) per mass of protein in the samples.

### IVIS Protocol

1. 20-24 hours after light (or TPCS₂ₐ injection), mice were given an intraperitoneal injection of 3 mg D-Luciferin (200 μl of 15 mg/ml stock).
2. After approximately 10 minutes the mice were anaesthetized by a subcutaneous injection of Zoletil (15 mg/kg xylasin, 7.5 mg/kg butorphanol, 9 mg/kg zolazepam, and 9 mg tiletamine)
3. 20 minutes after D-luciferin (Caliper Life Sciences) injection, the mice were placed in the IVIS instrument (IVIS Spectrum, model 124375R from PerkinElmer, Andor camera IS0825R4582; iKon Living Image version: 4.5.2.18424. Binning factor: 8; Excitation filter: Block; Emission filter: Open; f Number: 1) and pictures were taken with automatic exposure of Luminescence.
4. Bio-luminescence is expressed as “Total Flux” (photon/second)×10⁵.

### Results

FIG. 1A shows bio-luminescence imaging for the two injection sites for each of animals 1 to 6 (see Table 1). The sites subjected to PCI exhibited significantly stronger luminescence than the control sites receiving mRNA only. This was also reflected in the luciferase assay (FIG. 1B). The mean fold of increase for the PCI treated sites was 4.9 times higher than for the mRNA only sites. Illumination without the photosensitiser did not show the same improvement (data not shown).

### Example 2: Intradermal mRNA Delivery to BL/6 Mouse Skin In Vivo with Blue Light Illumination Various Times after mRNA/Photosensitiser Injection

Experiments were performed to study in vivo naked mRNA delivery to skin in mice using different contact times.

### Materials and Methods

The materials and methods were as used in Example 1, except that the administration was intradermal and blue light was used (wavelength between 400 and 540 nm with a peak at around 435 nm from the LumiSource illumination device produced by PCI Biotech AS, with a 13 mW/cm² fluence rate—a 6 minute illumination delivers 4.7 J/cm²). Luciferase activity was assessed as set out in Example 1 except that skin homogenates were used.

The administration and illumination protocols were as set out in the table below, Table 2.

TABLE 2
Administration and illumination protocols

| | | TPCS₂ₐ | mRNA | | |
|---|---|---|---|---|---|
| | | (μg per | (μg per | Illum. | |
| Group | | injection | injection | time point | No. of |
| no. | Name | site) | site) | (min) | animals |
| 1 | mRNA alone | — | 2 | — | 3 |
| 2 | PCI 0.003, | 0.003 | 2 | 10 | 3 |
| | 10 min | | | | |
| 3 | PCI 0.003, | 0.003 | 2 | 30 | 3 |
| | 30 min | | | | |
| 4 | PCI 0.003, | 0.003 | 2 | 60 | 3 |
| | 60 min | | | | |

### Results

The results of the luciferase assay are shown in FIG. 2. It is evident that a 10 minute contact time enhanced mRNA delivery by about 20 times, whereas previously used longer contact times were much less effective.

### Example 3: Intradermal mRNA Delivery to BL/6 Mouse Skin In Vivo with Blue Light Illumination 30 Seconds after mRNA/Photosensitiser Injection with Various Concentrations of Photosensitiser

Experiments were performed to study in vivo naked mRNA delivery to skin in mice using different photosensitiser doses with a short 30 second contact time.

### Materials and Methods

The materials and methods were as used in Example 2. Luciferase activity was assessed as set out in Example 1 except that skin homogenates were used.

The administration and illumination protocols were as set out in the table below, Table 3.

TABLE 3
Administration and illumination protocols

| | | TPCS₂ₐ | mRNA | | |
|---|---|---|---|---|---|
| | | (μg per | (μg per | | |
| Group | | injection | injection | Illum. | No. of |
| no. | Name | site) | site) | time point | animals |
| 1 | mRNA alone | — | 2 | — | 3 |
| 2 | PCI 0.05, | 0.05 | 2 | 30 seconds | 3 |
| | 30 seconds | | | | |
| 3 | PCI 0.01, | 0.01 | 2 | 30 seconds | 3 |
| | 30 seconds | | | | |
| 4 | PCI 0.003, | 0.003 | 2 | 30 seconds | 3 |
| | 30 seconds | | | | |

### Results

The results of the luciferase assay are shown in FIG. 3. The lower dose of photosensitiser (0.003 μg) was found to be more effective than higher doses even with the short contact time before illumination.

### Example 4: Intradermal mRNA Delivery to BL/6 Mouse Skin In Vivo with Blue Light Illumination 30 Seconds after mRNA/Photosensitiser Injection with Various Concentrations of Photosensitiser

Experiments were performed to study in vivo naked mRNA delivery to skin in mice using different photosensitiser doses with a short 30 second contact time.

### Materials and Methods

The materials and methods were as used in Example 3 but with even lower doses. Luciferase activity was assessed as set out in Example 1 except that skin homogenates were used.

The administration and illumination protocols were as set out in the table below, Table 4.

TABLE 4
Administration and illumination protocols

| | | TPCS₂ₐ | mRNA | | |
|---|---|---|---|---|---|
| | | (μg per | (μg per | | |
| Group | | injection | injection | Illum. | No. of |
| no. | Name | site) | site) | time point | animals |
| 1 | mRNA alone | — | 2 | — | 3 |
| 2 | PCI 0.01, | 0.01 | 2 | 30 seconds | 3 |
| | 30 seconds | | | | |
| 3 | PCI 0.001, | 0.001 | 2 | 30 seconds | 3 |
| | 30 seconds | | | | |
| 4 | PCI 0.0003, | 0.0003 | 2 | 30 seconds | 3 |
| | 30 seconds | | | | |

### Results

The results of the luciferase assay are shown in FIG. 4. The lower dose of photosensitiser (0.0003 μg) was found to be effective even with the short contact time before illumination.

### Example 5: Intradermal mRNA Delivery to BL/6 Mouse Skin In Vivo with Blue Light Illumination 30 Seconds after mRNA/Photosensitiser Injection

Experiments were performed to study in vivo naked mRNA delivery to skin in mice using a low photosensitiser dose with a short 30 second contact time.

### Materials and Methods

The materials and methods were as used in Example 2. Animals were injected at two sites (left or right). IVIS was examined as set out in Example 1.

The administration protocols were as set out in the table below, Table 5. The animals were illuminated with blue light 30 seconds after administration.

TABLE 5
Administration protocols

| Animal | Injection | Injection |
|---|---|---|
| no. | site A | site B |
| 1 | 2 μg mRNA alone | 2 μg mRNA + 0.003 μg |
| | | TPCS₂ₐ |
| 2 | 2 μg mRNA alone | 2 μg mRNA + 0.003 μg |
| | | TPCS₂ₐ |
| 3 | 2 μg mRNA alone | 2 μg mRNA + 0.003 μg |
| | | TPCS₂ₐ |
| 4 | 2 μg mRNA + 0.003 μg | 2 μg mRNA alone |
| | TPCS₂ₐ | |
| 5 | 2 μg mRNA + 0.003 μg | 2 μg mRNA alone |
| | TPCS₂ₐ | |
| 6 | 2 μg mRNA + 0.003 μg | 2 μg mRNA alone |
| | TPCS₂ₐ | |

### Results

FIG. 5A shows bio-luminescence imaging for the two injection sites for each of animals 1 to 6 (see Table 5). The mean results are shown in FIG. 5B. It can be seen that the sites subjected to PCI exhibited significantly stronger luminescence than the control sites receiving mRNA only (an increase of around 7.2 fold).

### Example 6: Intramuscular mRNA Delivery to BL/6 Mice In Vivo with Blue Light Illumination 30 Seconds after mRNA/Photosensitiser Injection with Different Photosensitiser Concentrations

Experiments were performed to study in vivo naked mRNA delivery to muscles in mice using different photosensitiser concentrations.

### Materials and Methods

The materials and methods were as used in Example 1, except that blue light was used (as in Example 2). IVIS was examined as set out in Example 1. Luciferase activity was assessed as set out in Example 1.

The administration and illumination protocols were as set out in the table below, Table 6.

TABLE 6
Administration and illumination protocols

| | | TPCS₂ₐ | mRNA | | |
|---|---|---|---|---|---|
| | | (μg per | (μg per | | |
| Group | | injection | injection | Illum. | No. of |
| no. | Name | site) | site) | time point | animals |
| 1 | mRNA alone | — | 2 | — | 4 |
| 2 | PCI 0.0003, | 0.0003 | 2 | 30 s | 4 |
| | blue 30 sec | | | | |
| 3 | PCI 0.0001, | 0.0001 | 2 | 30 s | 4 |
| | blue 30 sec | | | | |

### Results

FIG. 6A shows mean bio-luminescence at the injection sites for each group (see Table 6). FIG. 6B shows the results of the mean luciferase results for each group. It can be seen that the sites subjected to PCI exhibited significantly stronger luminescence and luciferase activity than the control sites receiving mRNA only, and that a low photosensitiser dose of 0.0001 μg was highly effective.

### Example 7: Intramuscular mRNA Delivery to BL/6 Mice In Vivo with Blue Light Illumination 30 Seconds after mRNA/Photosensitiser Injection with Different Photosensitiser Concentrations

Experiments were performed to study in vivo naked mRNA delivery to muscles in mice using different photosensitiser concentrations.

### Materials and Methods

The materials and methods were as used in Example 1, except that blue light was used (as in Example 2). Luciferase activity was assessed as set out in Example 1. The administration and illumination protocols were as set out in the table below, Table 7.

TABLE 7
Administration and illumination protocols

| | | TPCS₂ₐ | mRNA | | |
|---|---|---|---|---|---|
| | | (μg per | (μg per | | |
| Group | | injection | injection | Illum. | No. of |
| no. | Name | site) | site) | time point | animals |
| 1 | mRNA alone | — | 2 | — | 3 |
| 2 | PCI 0.1, | 0.1 | 2 | 30 s | 3 |
| | blue 30 sec | | | | |
| 3 | PCI 0.003, | 0.003 | 2 | 30 s | 3 |
| | blue 30 sec | | | | |
| 4 | PCI 0.0003, | 0.0003 | 2 | 30 s | 3 |
| | blue 30 sec | | | | |

### Results

The results of the luciferase assay are shown in FIG. 7. The lower dose of photosensitiser (0.0003 μg) was found to be effective even with the short contact time before illumination.

### Example 8: Intramuscular mRNA Delivery to BL/6 Mice In Vivo with Blue Light Illumination 10 Minutes after mRNA/Photosensitiser Injection with Different Photosensitiser Concentrations

Experiments were performed to study in vivo naked mRNA delivery to muscles in mice using different photosensitiser concentrations.

### Materials and Methods

The materials and methods were as used in Example 1, except that blue light was used (as in Example 2). Luciferase activity was assessed as set out in Example 1.

The administration and illumination protocols were as set out in the table below, Table 8.

TABLE 8
Administration and illumination protocols

| | | TPCS₂ₐ | mRNA | | |
|---|---|---|---|---|---|
| | | (μg per | (μg per | Illum. | |
| Group | | injection | injection | time point | No. of |
| no. | Name | site) | site) | (min) | animals |
| 1 | mRNA alone | — | 2 | — | 3 |
| 2 | PCI 0.1, | 0.1 | 2 | 10 | 3 |
| | 10 min | | | | |
| 3 | PCI 0.003, | 0.003 | 2 | 10 | 3 |
| | 10 min | | | | |
| 4 | PCI 0.0003, | 0.0003 | 2 | 10 | 3 |
| | 10 min | | | | |

### Results

The results of the luciferase assay are shown in FIG. 8. The lower dose of photosensitiser (0.0003 μg) was found to be more effective than the higher doses.

### Example 9: Intramuscular mRNA Delivery to BL/6 Mice In Vivo with Red Light Illumination 5 or 10 Minutes after mRNA/Photosensitiser Injection with Different Light Doses

Experiments were performed to study in vivo naked mRNA delivery to muscles in mice using different light doses concentrations and intervals before illumination.

### Materials and Methods

The materials and methods were as used in Example 1 except that different light doses were used as described below. Luciferase activity was assessed as set out in Example 1.

The administration and illumination protocols were as set out in the table below, Table 9. Two sets of experiments were conducted using either a 5 or 10 minute interval after administration and before illumination.

TABLE 9
Administration and illumination protocols

| | | TPCS₂ₐ | mRNA | | | |
|---|---|---|---|---|---|---|
| | | (μg per | (μg per | | | |
| Group | | injection | injection | Illum. | | No. of |
| no. | Name | site) | site) | dose | | animals |
| 1 | mRNA alone | — | 2 | — | | 3 |
| 2 | PCI 0.0001, IM | 0.0001 | 2 | 0.3 | J/cm² | 3 |
| | red 10 min, | | | | | |
| | 0.3 J | | | | | |
| 3 | PCI 0.0001, IM | 0.0001 | 2 | 1 | J/cm² | 3 |
| | red 10 min, | | | | | |
| | 1 J | | | | | |
| 4 | PCI 0.0001, IM | 0.0001 | 2 | 3 | J/cm² | 3 |
| | red 10 min, | | | | | |
| | 3 J | | | | | |

### Results

The results of the luciferase assay are shown in FIG. 9. With a 5 minute interval and a TPCS₂ₐ dose of 0.0001 μg an 8-9 fold increase by PCI was observed over a red light dose range of 0.3 to 3 J/cm² (FIG. 9A). A 10 minute interval was also effective but not at the same levels as the 5 minute interval and seemed to increase with light dose from 0.3 to 3 J/cm² (FIG. 9B).

### Example 10: Intramuscular mRNA Delivery to BL/6 Mice In Vivo with Red Light Illumination 5 Minutes after mRNA/Photosensitiser Injection Using Very Low Photosensitiser Concentrations

Experiments were performed to study in vivo naked mRNA intramuscular delivery to mice using very low photosensitiser doses after a 5 minute contact time.

### Materials and Methods

The materials and methods were as used in Example 1. Animals were injected at two sites (left and right thigh). IVIS was examined as set out in Example 1.

The administration protocols were as set out in the table below, Table 10. The animals were illuminated with red light 5 minutes after administration (1 J/cm²).

TABLE 10
Administration and illumination protocols
Experiment 1:

| | | TPCS₂ₐ | | mRNA | | |
|---|---|---|---|---|---|---|
| | | (μg per | | (μg per | Illum. | |
| Group | | injection | | injection | time | No. of |
| no. | Name | site) | | site) | point | animals |
| 1 | mRNA alone | — | | 2 | — | 3 |
| 2 | PCI 0.0003, IM | 0.0003 | (0.3 ng) | 2 | 5 min | 3 |
| | red 5 min | | | | | |
| 3 | PCI 0.0001, IM | 0.0001 | (0.1 ng) | 2 | 5 min | 3 |
| | red 5 min | | | | | |
| 4 | PCI 0.00001, IM | 0.00001 | (0.01 ng) | 2 | 5 min | 3 |
| | red 5 min | | | | | |

Experiment 2:

| | | TPCS₂ₐ | | mRNA | | |
|---|---|---|---|---|---|---|
| | | (μg per | | (μg per | Illum. | |
| Group | | injection | | injection | time | No. of |
| no. | Name | site) | | site) | point | animals |
| 1 | mRNA alone | — | | 2 | — | 3 |
| 2 | PCI 0.0001, IM | 0.0001 | (0.1 ng) | 2 | 5 min | 3 |
| | red 5 min | | | | | |
| 3 | PCI 0.00003, IM | 0.00003 | (0.03 ng) | 2 | 5 min | 3 |
| | red 5 min | | | | | |
| 4 | PCI 0.000003, IM | 0.000003 | (0.003 ng) | 2 | 5 min | 3 |
| | red 5 min | | | | | |

### Results

FIG. 10 shows the mean results of bio-luminescence imaging for the two injection sites for the animals in each group for experiments 1 and 2 (FIGS. 10A and B, respectively). With a 5 minute interval a 5- and 7-fold increase was observed at TPCS₂ₐ doses of 0.0001 and 0.0003 μg, respectively. An effect was observed even when using TPCS₂ₐ at a very low dose of 0.000003 μg (FIG. 10B).

### CONCLUSIONS FROM EXAMPLES

Good correspondence between the IVIS and luciferase assay results was observed. The fold increase (PCI/mRNA alone) with illumination only 30 seconds after mRNA/photosensitiser injection is higher than has been achieved in earlier experiments in which illumination was conducted 60 minutes after injection (3-5 times improvement). The improvement relative to using no photosensitiser is in the order of 30 fold. Shorter contact times were also effective at very low photosensitiser dose allowing for reduced side effects in subjects.

1. An in vivo method for introducing an mRNA molecule into the cytosol of a cell(s) in a subject, said method comprising
i) contacting said cell(s) with an mRNA molecule and a photosensitising agent, wherein said mRNA is naked, and
ii) irradiating the cell(s) with light of a wavelength effective to activate the photosensitising agent,
wherein said photosensitising agent is a sulphonated meso-tetraphenyl chlorin, sulfonated tetraphenylporphine or a di- or tetrasulfonated aluminium phthalocyanine used in an amount of 0.000001-0.001 μg and said contacting step is performed for 30 seconds to 10 minutes.

2. The method as claimed in claim 1 wherein said photosensitising agent is TPCS₂ₐ or a pharmaceutically acceptable salt thereof.

3. The method as claimed in claim 1 wherein said photosensitising agent is used in an amount of 0.0001-0.0003 μg.

4. The method as claimed in claim 1 wherein said photosensitising agent is used at a concentration of 0.0005 to 1 μg/ml, preferably 0.005 to 0.5 μg/ml.

5. The method as claimed in claim 1 wherein the mRNA molecule is from 50 to 10,000 nucleotides long.

6. The method as claimed in claim 1 wherein the mRNA is used in an amount of 0.1 to 100 μg, preferably at a concentration of 5 to 5000 μg/ml.

7. The method as claimed in claim 1 wherein said mRNA is expressed in said cell(s), wherein preferably the polypeptide expressed by said mRNA is a therapeutic molecule, preferably an antibody, a vaccine polypeptide or a cytotoxic molecule.

8. The method as claimed in claim 1 wherein said cell(s) is a mammalian cell(s) or a fish cell(s).

9. The method as claimed in claim 1 wherein the light has a wavelength of 400-475 nm, preferably 400-435 nm, or a wavelength of 620-750 nm, preferably 640-660 nm.

10. The method as claimed in claim 1 wherein said contacting step is performed for 30 to 60 seconds.

11. The method as claimed in claim 1 wherein the cell(s) is irradiated for between 15 seconds and 60 minutes, preferably for 0.5 to 12 minutes, preferably for 4 to 6 minutes.

12. The method as claimed in claim 1 wherein the cell(s) is irradiated with a light dose of from 0.01 to 50 J/cm², preferably 0.3 to 3 J/cm².

13. The method as claimed in claim 1 wherein said cell(s) is contacted with said mRNA and photosensitising agent simultaneously, separately or sequentially, preferably simultaneously.

14. The method as claimed in claim 1 wherein said subject is a mammal or a fish, preferably the mammal is a monkey, cat, dog, horse, donkey, sheep, pig, goat, cow, mouse, rat, rabbit or guinea pig, most preferably the subject is a human.

15. The method as claimed in claim 1 wherein said mRNA and/or said photosensitising agent is administered locally, preferably intradermally, intramuscularly or intratumourally, preferably by injection.

16. An in vivo method of expressing a polypeptide in a cell(s) in a subject, comprising introducing an mRNA molecule into a cell(s) of the subject by the method as defined in claim 1, wherein said mRNA molecule encodes said polypeptide.

17. A pharmaceutical composition comprising an mRNA molecule and a photosensitising agent, wherein said mRNA is naked and said photosensitising agent is a sulphonated meso-tetraphenyl chlorin, sulfonated tetraphenylporphine or a di- or tetrasulfonated aluminium phthalocyanine and is provided in the amount of 0.000001 to less than 0.0001 μg, wherein preferably said photosensitising agent is:
i) TPCS₂ₐ or a pharmaceutically acceptable salt thereof; or
ii) used at a concentration of 0.0005 to 1 μg/ml, preferably 0.005 to 0.5 μg/ml;
and/or said mRNA:
i) is from 50 to 10,000 nucleotides long;
ii) is used in an amount of 0.1 to 100 preferably at a concentration of 5 to 5000 μg/ml;
or
iii) expresses a polypeptide which is a therapeutic molecule, preferably an antibody, a vaccine polypeptide or a cytotoxic molecule.

18-20. (canceled)

21. A method of treating or preventing a disease, disorder or infection in a subject comprising introducing an mRNA molecule into one or more cells in vivo in said subject according to the method as defined in claim 1.

22-25. (canceled)

26. The method of treating or preventing a disease, disorder or infection as claimed in claim 21, wherein:
i) said disease, disorder or infection is one which would benefit from expression of one or more polypeptides, preferably for protein therapy, immunotherapy or gene therapy;
ii) an immune response is generated to said expressed polypeptide, preferably said treatment or prevention occurs via vaccination, wherein preferably said vaccination is prophylactic or therapeutic;
iii) said disease is cancer or said infection is a viral or bacterial infection; and/or
iv) said mRNA and photosensitising agent is administered intradermally, intramuscularly or intratumourally.

27. The method of treating or preventing a disease, disorder or infection as claimed in claim 21 wherein:
i) said photosensitising agent is TPCS₂ₐ or a pharmaceutically acceptable salt thereof;
ii) said photosensitising agent is used in an amount of 0.0001-0.0003 μg;
iii) said contacting step is performed for 30 to 60 seconds;
iv) the cell(s) is irradiated for between 15 seconds and 60 minutes, preferably for 0.5 to 12 minutes, preferably for 4 to 6 minutes; and/or
v) the cell(s) is irradiated with a light dose of from 0.01 to 50 J/cm², preferably 0.3 to 3 J/cm².
